(19) United States
(12) Patent Application Publication
(10) Pub. No.: US 2022/0168094 A1
(43) Pub. Date: Jun. 2, 2022

(54) Intraocular Lens Combination for Restoration of Refraction and Accommodation

(71) Applicant: AKKOLENS INTERNATIONAL B.V., Breda (NL)

(72) Inventors: Michiel Christiaan Rombach, Breda (NL); Willem Pieter Van Lawick, Breda (NL)

(21) Appl. No.: 17/442,743

(22) PCT Filed: Mar. 24, 2020
(86) PCT No.: PCT/NL2020/050195

§ 371 Date: Sep. 24, 2021

(30) Foreign Application Priority Data
Mar. 25, 2019 (NL) 2022797
Apr. 1, 2019 (NL) 2022847

(51) Int. Cl. A61F 2/16

(52) U.S. Cl. CPC A61F 2/1618; A61F 2/1621

(57) ABSTRACT

An intraocular lens combination of independent lens constructions includes a first lens construction for restoration of refraction of the aphakic eye and a second lens construction for restoration of accommodation of the phakic eye. The preferred embodiment of the first lens construction includes a lens of fixed optical power implanted in the capsular bag and second lens construction including an accommodative lens of variable optical power implanted in front of the bag. The intraocular lens combination can include corrective optics to correct for both fixed and variable residual optical errors.

### CROSS-REFERENCE TO RELATED APPLICATIONS

This application is the United States national phase of International Application No. PCT/NL2020/050195 filed Mar. 24, 2020, and claims priority to The Netherlands Patent Application Nos. 2022797 filed Mar. 25, 2019 and U.S. Pat. No. 2,022,847 filed Apr. 1, 2019, the disclosures of which are hereby incorporated by reference in their entirety.

### BACKGROUND OF THE INVENTION

### Field of the Invention

An intraocular lens combination of independent lens constructions includes a first lens construction for restoration of refraction of the aphakic eye and a second lens construction for restoration of accommodation of the phakic eye. The preferred embodiment of the first lens construction includes a lens of fixed optical power implanted in the capsular bag and second lens construction including an accommodative lens of variable optical power implanted in front of the bag. The intraocular lens combination can include corrective optics to correct for both fixed and variable residual optical errors.

### Description of Related Art

Accommodative intraocular lenses restore accommodation of the human eye, meaning, provide the retina with a sharp focus at any object distance, from far to reading distance, by translation of the focus the incoming light beam along the optical axis.

Accommodative lenses can vary the focal distance by movement of the lens along the optical axis, for example, movement of single fixed focus lenses in the eye, as disclosed in, for example, US2019053893 and WO2006NL50050 (EP1871299), or, alternatively, movement of multiple lenses along the optical axis, as disclosed in, for example, US2018221139 and US2013013060 (CA2849167, US2002138140). Such lens movements can be driven by the ciliary muscle, generally via the remains, the rim, of the capsular bag, as in US2019053893, or, alternatively, such movement can be driven by the iris, as in, for example, WO2019027845, ES2650563 and US2008215146, or, alternatively, such movement can be driven by the zonulae which connect the capsular bag to the ciliary mass of the eye, as in, for example, US2018353288.

Alternatively, a single multifocal lens, for example, a lens with a single cubic free-form surface or, alternatively, a lens with a bifocal or multifocal optical surface, can be moved in a direction perpendicular to the optical axis, as in US201010624.

Also, translation of the focus of a lens along the optical axis can be achieved by a change of lens shape, a increase of radial thickness of the lens, along the optical axis, as in, for example, AU2014236688, US201562257087 and US2018256315, which discloses lenses in which an elastic container filled with a fluid comprises a variable lens, or, alternatively, as in US2018344453, U.S. Ser. No. 10/004,595, US2018271645, US2019015198 and U.S. Pat. No. 9,744,028 which disclose a change in shape of a uniform elastic lens and, alternatively, as in US2019000162 which discloses an elastic lens driven by fluid pressure of the vitreous of the eye. US2012310341, US2011153015 and DE112009001492 disclose any type of shape changing lenses which lenses are positioned at the sulcus plane, instead of inside the remains of the capsular bag of the eye with such change of shape driven directly by the ciliary mass or zonulae system of the eye, or, alternatively, by the iris, or, alternatively, by the sclera, for example by the sulcus tip connected to the sclera of the eye.

In addition, variable optics can be provided by two optical elements with each element comprising at least one free-form optical surface with such a shape that the combination of these shapes provides a variable lens of which the optical power depends on the relative position of the elements in a direction perpendicular to the optical axis, as in, for example, EP1720489, with the optical elements connected by, for example, a mechanical connector, as in NL2015644, or by glueing, or, by repolymerization by monomers from which the lens material also originates. Such lenses can provide a non-linear variation on optical power in response to a linear change in the mutual position of the optical elements, as in, for example, NL2012133, with the said free-form optical surfaces distributed over any number of surfaces of the optical elements, as in NL2012420. Intraocular lenses comprising such free-form variable optics and their application are known from, for example, but not restricted hereto from, referring to such intraocular lenses and applications: WO2019022608 disclosing free-form surfaces of, for example, different Zernike orders, which algorithms can also be expressed by, for example, NURBS or spline algorithms;

US2012323320 discloses such mechanically adjustable lenses and US2017312133 discloses such laser adjustable lenses; NL2015538 and US2014336757 disclose haptics for the sulcus plane; NL2015616 discloses irrigation channels to reduce increase in intraocular pressure; US2016030162 discloses an electricity generator driven by such lens; WO 2009051477 discloses piggy back, thin lens elements added to main lens elements to correct for residual optical errors; US2014074233 and U.S. Pat. No. 9,744,028 disclose partly anchoring of such lenses in the remains of the capsular bag; US2012257278 and EP1932492 disclose principles of variable correction of any combination of variable aberrations; WO2014058316 discloses alternative shapes for the elastics haptics of such lenses; NL210980 and EP2765952 disclose customized optics of such lenses; NL2009596 discloses mechanical additions to such lens to protect the posterior surface of the iris of the eye.

Note that translation of the focus of a lens along the optical axis can be a parallel mutual shift of optical elements as used as the main example of variable lenses in this document, but also a rotation of at least one element as in the rotation of optical elements comprising at least two chiral optical surfaces in a direction perpendicular to the optical axis, WO2014058315 and ES2667277, or, alternatively, a combination of wedging and rotation of at least two complex free-form surfaces, for example adapted cubic optical surfaces, as in, for example, US2012323321.

All the references cited in this document are considered part of this document as well as other documents herein not referred to which documents cover any disclosures which are related to the disclosures by all the references cited in this document.

### SUMMARY OF THE INVENTION

The present document, the present invention, discloses an invention concerning an intraocular lens combination of at least two lens constructions with the combination including at least one first lens construction to provide restoration of refraction of the eye and at least one second lens construction to provide restoration of accommodation of the eye. Said lens constructions can be independent, meaning said lens constructions being separate lens constructions.

### BRIEF DESCRIPTION OF THE DRAWINGS

FIG. 1. A schematic cross section of the human eye with the optical axis of the eye, 1, the cornea, 2, a surgical incision in the cornea, 2a, the anterior chamber of the eye, 3, and the posterior chamber of the eye, 3a, the iris, 4, the sulcus, 5, the ciliary mass, 6, the zonulae connection the ciliary mass to the capsular bag, 7, the natural lens of the eye, 8, representing the first lens construction, the capsular bag containing the natural lens, 9, and the retina, 10. This figure also shows a second lens construction, in this example a elastic lens construction, 11, which lens changes optical power by change in at least one radius of an optical surface in a direction, 12b, along the optical axis, driven by contraction or relaxation, 12a, of the ciliary muscle of the eye.

FIG. 2 (for anatomical and lens structures refer also to FIG. 1). The natural lens of the eye, in this example the natural lens representing the first lens construction, a second lens construction, in this example a lens construction comprising two optical elements which both translate in a direction perpendicular to the optical axis, 13, with haptics, 14, which translate the movement of the ciliary muscle into mutual movement of the optical elements of the lens, which lens comprises at least two free-form optical surfaces and changes optical power by translation of at least one of the optical elements direction, 15, perpendicular to the optical axis, which direction is in parallel to the direction of contraction/relaxation of the ciliary muscle along the optical axis driven by contraction or relaxation, 12a, of the ciliary muscle of the eye.

FIG. 3 (For anatomical and lens structures refer also to FIG. 1-2). The natural lens of the eye, representing the first lens construction, a second lens construction, in this example a lens construction comprising two optical elements of which only one element, 16, comprising a solid, 17, and a elastic haptic, 18, (an haptic combination for a single optical element as illustrated in, for example, WO2006NL50050, FIG. 7 and US2010106245, FIG. 2) which element translates in a direction perpendicular to the optical axis, with the other element being a static element, 19, in this example an optical surface added to, enscribed in (by Smile laser procedures) or onto (by Lasik laser procedures), the cornea of the eye, by, for example, a surgical laser. The translating optical element moves in a direction perpendicular to the optical axis. Note that movement/translation perpendicular to the optical axis includes all such movements, including, but not restricted to, lateral translations, shifts, rotations, and wedgings.

FIG. 4 (for anatomical and lens structures refer also to FIG. 1-3). This figure shows the preferred embodiment of the inventions disclosed in the present document with an artificial first lens construction, 22, for example, a standard monofocal intraocular lens, to provide restoration of refraction of the eye following removal of the natural lens, with the first lens construction surgically implanted though a capsulorhexis, a hole, 23a, in the capsular bag of which the posterior part and rim remains, 23, in combination with a second lens construction, 21, as outlined in FIG. 2, with elements mutually translating in a direction perpendicular to the optical axis.

FIG. 5 (for anatomical and lens structures refer also to FIG. 1-4). In The embodiment of the first lens construction is a, for example, standard monofocal lens to which monofocal optical surfaces a free-form surface, 25, is added which free-form surface provides, in combination with a complementary free-form surface, 24, form a lens of variable power of which the power depend on the mutual shift of surface 25 and 25 with such shift provide by only the second lens construction.

FIG. 6. (for anatomical and lens structures refer also to FIG. 1-5). In this embodiment the first lens construction is a, for example, standard monofocal lens and the second lens construction distributed over a translating optical element, 26 and a static optical surface, 27, comprised by the cornea, for example, enscribed to the cornea by a surgical laser, as also outlined in FIG. 3. To any optical surface additional optical surfaces can be added which surfaces can provide correction of any residual aberration, in this example a single such surface, 28, is added to the laser enscribed optical surface on the cornea.

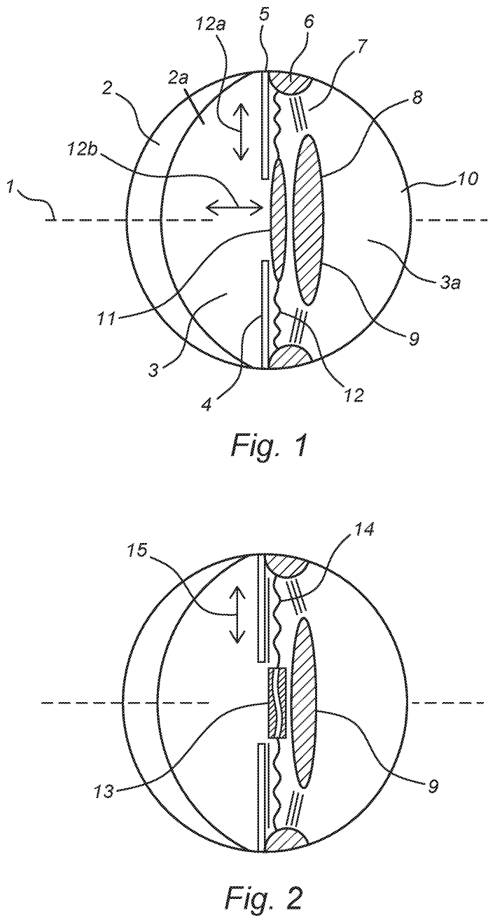

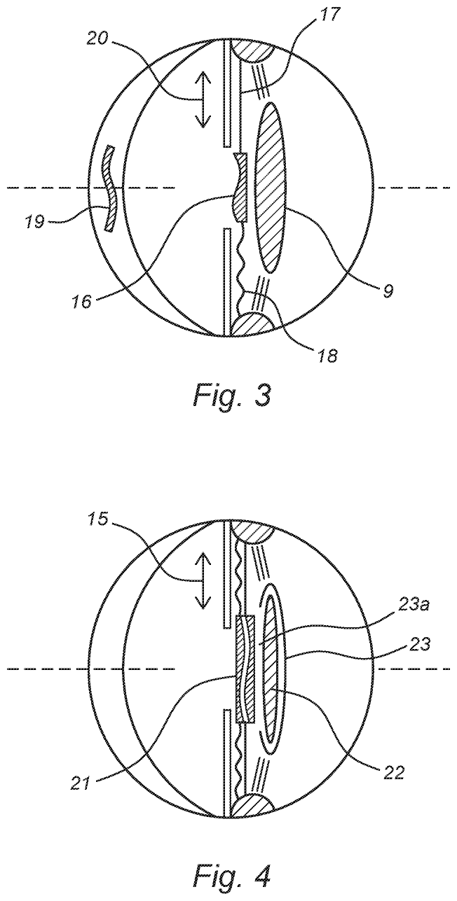

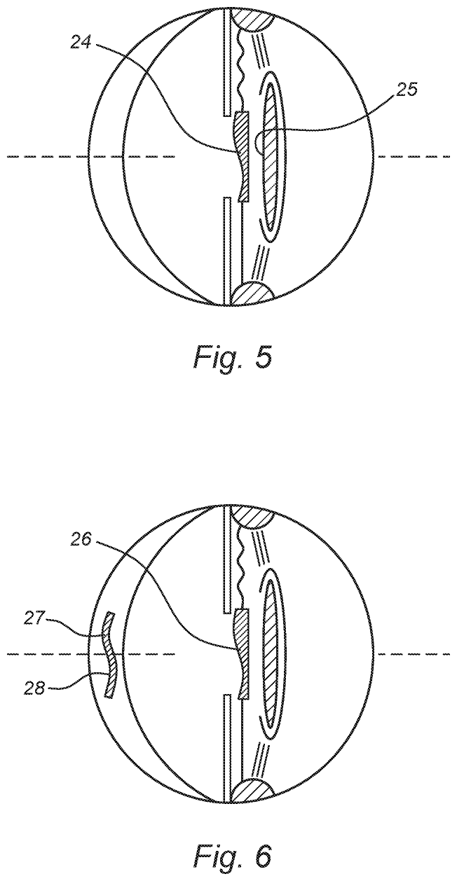

### DESCRIPTION OF THE INVENTION

The combination can also comprise additional corrective optics to correct for any fixed undesired aberrations such as, for example, astigmatism of the cornea. Such corrective optics can be added to either the first lens construction or, alternatively, to the second lens construction or, alternatively, the optics can be distributed over both construction. The combination can also comprise additional corrective optics to correct for any variable undesired aberrations, for example, undesired variable astigmatic aberration. These corrective optics can be added to, for example, the second lens construction.

Furthermore, additional corrective optics can be added to the second construction which corrective optics add desired variable aberrations, for example, add variable aspherical aberration to support sharp vision to support near, for example, reading, vision.

All patents and published patent applications identified below in this detailed description of the invention are hereby incorporated by reference.

The first lens construction, the refractive construction, can comprise at least one optical component to provide fixed optical power to restore refraction of, for example, an aphakic eye, meaning an eye from which the natural lens is surgically removed, with the removal due a cataract of the eye or, alternatively, to clear lens extraction, CLE, meaning removal of a transparent lens due to, for example presbyopia and/or severe myopia. Note that the natural lens of the eye can also be considered, in rare cases, as a first lens construction.

Generally, the first lens construction is any artificial intraocular lens construction implanted into the eye, for example, a monofocal intraocular lens, or, alternatively, a multifocal intraocular lens implanted in, for example, the capsular bag of the eye, or, alternatively, any lens implanted in the eye, for example in the anterior chamber of the eye. Such first lens construction is generally implanted in the posterior chamber of the eye, in the remains, in the rim, of the capsular bag of the eye.

The second lens construction, the accommodative construction, comprises at least one optical component to provide variable optical power to restore accommodation of the phakic eye, meaning adding accommodation to a refractive lens of fixed optical power, for example to a first lens construction such as an artificial monofocal lens. So, an accommodation range of, for example, 0-4D can be added by the second lens construction to a fixed refraction of, say, 20D provided by the first lens construction.

Firstly, such second lens construction can comprise a combination of at least two optical elements comprising a combination of at least two free-form optical surfaces with each optical element comprising at least one free-form optical surface with the combination providing variable defocus power of which the power depends on the degree of mutual translation of the optical elements in a direction perpendicular to the optical axis of the eye as disclosed also in, for example, EP1720489.

Such second lens constructions must comprises mechanical components to provide translation of lateral compression of the construction into mutual translation of the optical elements as disclosed in, for example, US2010106245 and multiple other documents cited above.

Secondly, a second construction can comprise at least one elastic optical component which component provides variable defocus power which power depends on the degree of change of shape of the elastic optical component as disclosed in, for example, but not limited hereto, documents US2011153015 and US2019015198. Such second construction also comprises mechanical components to provide translation of lateral compression of the construction into a change of shape of the elastic optical component. The elastic optical component can be made of uniform elastic material, as in, for example, DE11200900492 or, alternatively, can be uniform elastic material, for example, a fluid, into an elastic lens shaped container or elastic lens shaped casing, as in, for example, but not restricted hereto, AU2014236688. Thirdly, a second construction can comprise any optical component which component provides variable defocus power which power depends on the degree of any changes of the optical component. Such second construction also comprises mechanical components to provide translation of lateral compression of the construction into a change of the optical component.

Second lens constructions are adapted to be implanted at the sulcus plane or ciliary plane of the eye, meaning in front of, anterior of, the capsular bag of the eye and comprise at least one mechanical component providing translation of movement of the ciliary mass or zonulae or any other related anatomical structure of the eye into mutual translation of the optical elements or, alternatively, in a change of shape of the elastic optical element or in any change of an optical element or optical component.

The second lens construction can also comprise at least one additional optical surface to provide corrective optical power to correct at least one optical aberration of the eye. For example, fixed optical power can correct a fixed power aberration, for example residual refractive error of the eye such as myopia, hyperopia or astigmatism of the eye, or any combination of such fixed power aberrations.

The second lens construction can also comprise at least one additional optical surface to provide variable optical power to correct at least one, undesired, variable optical aberration of the eye other than the, desired, variable defocus. Such undesired variable aberration can be, for example, but not restricted hereto, variable aspherical aberration, or, variable astigmatism, or, variable coma, or, variable trefoil aberrations, or, any combination of any variable aberrations.

The posterior surface of the second lens construction can be shaped such, for example, can be shaped as a negative, concave, lens which shape is adapted to provide a fit with the convex shape of the anterior surface of the first lens construction, or, alternatively, both surfaces can be planar with the desired optical power of the first lens construction concentrated on the posterior surface of the first lens construction. Such shapes can allow for desired improved movement of the constructions.

So, the present document discloses an intraocular lens combination with a first lens construction and a second lens construction which lens combination provides fixed optical power and variable optical power.

The first lens construction provides at least a part of the fixed optical power of the lens combination and that the second lens construction provides at least a part of the variable optical power of the lens combination. The fixed optical power restores the refraction of the aphakic eye, meaning replaces the optical power of the natural lens to allow the eye to focus sharp at far distance. The variable power provides additional optical power to also allow the eye to focus at near distances, allows the eye to accommodate.

The first lens construction generally includes a monofocal intraocular lens and implanted inside the capsular bag of the eye, and, the second lens construction is a variable lens construction to provide variable optical power and implanted outside, in front of, the capsular bag of the eye.

The first lens construction can provide all fixed optical power of the lens combination with the second lens construction providing all variable optical power of the lens combination, or, alternatively, the first lens construction can provide a part of the variable optical power of the lens combination with the second lens construction providing a part of the fixed optical power of the lens combination.

The lens constructions can remain independent, meaning separate constructions in the eye. However, the constructions can also be connected, in the eye, by any connection component, for example, any pin-in-hole, groove-in-groove or other mechanical connection, or, alternatively, any biocompatible glue or repolymerization process. Such connection will provide rotational and tilt stability to the second lens construction because the first lens construction is generally well stabilized inside the remains of the capsular bag.

The first lens construction can be a monofocal lens construction implanted at any position in the eye, with the preferred position being apposition inside the remains of the capsular bag following explanation of the natural lens of the eye. The first lens construction can comprise a single lens, for example a basic spherical lens, or, alternatively, a multifocal lens, for example a bifocal lens to provide at least one fixed optical power to provide restoration of refraction of an aphakic eye and the second lens construction a single lens, for example, for example a basic spherical lens, or, alternatively, a multifocal lens, for example a bifocal lens to provide at least one fixed optical power with the combination of spherical lenses providing variable optical power of the intraocular lens combination.

The second lens construction is a variable lens construction to provide all variable optical power, or, alternatively, part of the variable power, or, alternatively, the second lens construction comprises at least one free-form optical surface which surface provides a variable lens in combination with at least one another such free-form surface which another free-form surface is not included in the second lens construction, but, for example, is included as an optical components of the first lens construction, or, alternatively, is included in any other intraocular construction, or, alternatively, is added to the cornea of the eye by laser surgery.

The second lens construction, the variable lens construction, can comprise a combination of at least two optical elements comprising a combination of at least two free-form optical surfaces with each optical element comprising at least one free-form optical surface with the combination adapted to provide variable defocus power of which the power depends on the degree of mutual translation of the optical elements in a direction perpendicular to the optical axis of the eye. Such accommodating lenses are known from, for example, EP1720489, NL2015644, NL2012133, NL2012420 and NL2009596 and many related documents hereto. The second lens construction should also comprise mechanical components, haptics, adapted to provide translation of lateral compression of the construction into mutual translation of the optical elements. The second lens construction can also comprise at least one additional optical surface to provide corrective optical power to correct at least one optical aberration of the eye, for example, provide fixed optical power to correct at least one fixed optical aberration of the eye, which can be a residual refractive error of the eye, or, alternative, which can be myopia, hyperopia or astigmatism of the eye. Also, the additional optical surface provide variable optical power to correct at least one variable optical aberration of the eye other than variable defocus, for example, undesired variable aspherical aberration, or add the same, in case this is desired. The residual refractive error of the eye can be myopia of the eye, or, hyperopia of the eye, or, astigmatism of the eye, with additional optical surface providing variable optical power to correct at least one variable optical aberration of the eye other than variable defocus, for example, the variable optical aberration of the eye is variable aspherical aberration.

The shape of the posterior optical surface of the second lens construction can be adapted to provide a fit with the anterior surface of the first lens construction to support proper movement of any component of the second lens construction, or, alternatively, to prevent any movement, for example, decentration of the first lens construction. For example, a concave optical surface can be added to the posterior surface of the second lens construction which surface can be compensated for a an added convex optical surface to the anterior surface of the first lens construction such that said surfaces provide support for centration of the second lens construction versus the optical axis of the eye.

Such accommodating second lens constructions are, preferably, implanted at the sulcus plane, or, alternatively, in the sulcus of the eye, and driven directly by the ciliary mass/zonula system so that posterior capsular opafication, PCO, or shrinkage of the capsular bag will not affect the accommodative properties of the lens construction. Alternatively, The second lens construction, the variable lens construction, can comprise at least one elastic optical component adapted to provide variable defocus power which power depends on the degree of change of shape of the elastic optical component. Such components are known from AU2014236688, U.S. Pat. No. 1,011,745 and US2018256311, which documents disclose a lens shaped elastic container filled with a fluid or an elastic lens adapted to be implanted inside the remains of the capsular bag. US2019000612 discloses such lenses adapted to be implanted at the sulcus plane, in front of the capsular bag. So, the second lens construction can comprise at least one elastic optical component which component provides variable defocus power which power depends on the degree of change of shape of the elastic optical component and comprises mechanical components, haptics, adapted to provide translation of lateral compression of the construction into a change of shape of the elastic optical component. Such second lens constructions are preferably implanted at the sulcus plane of the eye and comprise at least one mechanical component providing translation of movement of any anatomical structure of the eye, for example the ciliary mass of the eye, into mutual translation of the two optical elements, or, alternatively, into a change of shape of the elastic optical component. So, the second lens construction should comprises at least one mechanical component to provide translation of movement of the ciliary mass of the eye into mutual translation of the optical elements, or, alternatively, the second lens construction should comprise at least one mechanical component to provide translation of movement of the ciliary mass of the eye into a change of shape of the elastic optical component.

At least one of the lens constructions can also comprises at least one additional optical surface provide corrective optical power to correct at least one residual optical aberration of the eye. Such corrections can be corrected for by the first lens construction, for example, severe corrections present in the eye pre-operative, for example, severe astigmatism due to cornea aberrations. Alternatively, corrections can be provided by the second lens construction after implantation of the, likely large, first lens construction, of which the surgery can introduce additional aberrations of the eye. A method for such corrections is outlined in the section on methods outlined below in this document.

A method for implantation of a lens combination including a first lens construction and a second lens construction with the first lens construction providing at least a part of the fixed optical power of the lens combination and the second lens construction providing at least a part of the variable optical power. The procedure, the method, for implantation can be implantation of both the first and second constructions during the same surgery. However, the method can also comprise multiple surgical steps including, firstly, replacement of the natural lens by a first lens construction, for example, a monofocal lens, as in, for example, standard cataract surgery, and secondly, after a period of time post operative, evaluation of residual fixed and variable aberrations of the eye and, thirdly, implantation of a second, customized, lens construction adapted to provide a combination of accommodation and correction of any residual optical aberrations due to any optical characteristic of the particular eye and/or due to optical characteristic of the first lens construction and/or to the particular position to which the first lens construction has settled inside the eye. Such method can be designed to correct multiple residual refractive and other fixed optical aberrations and variable optical aberrations. The implantation of the second lens construction is preferably before the corneal incision of the first implantation has fully healed so that no undesired aberrations are introduced by additional corneal incisions.

However, the optical functions of restoration of refraction of the aphakic eye and accommodation of the phakic eye en correction of any residual optical aberrations can also be distributed over the first lens construction and the second lens construction. Such distribution mainly applies to intraocular lens combinations comprising a second lens construction which construction comprises combination of at least two optical elements comprising a combination of at least two free-form optical surfaces with each optical element comprising at least one free-form optical surface with the combination adapted to provide variable defocus power of which the power depends on the degree of mutual translation of the optical elements in a direction perpendicular to the optical axis of the eye. For example, the first lens construction can comprises a combination of at least one optical component adapted to provide fixed optical power to provide restoration of refraction of an aphakic eye and at least one free form optical surface which, in combination with at least one complementary free-form surface provides a lens which provides variable defocus power of which the power depends on the degree of mutual translation of the optical elements in a direction perpendicular to the optical axis of the eye. Such first lens construction can be combined with a second lens construction comprising one optical element comprising the complementary free-form surface. Such first lens construction can be implanted in a stable position in the eye, for example in the capsular bag, or, alternatively, in the anterior chamber, with a stable position meaning a position in which the construction is intended not to translate. Or, alternatively, The first lens construction can comprise a standard monofocal lens, the second lens construction a single free-form surface, with for example, a mechanical design as in EP1871299 and US2010106245 with the complementary free-form surface added on the cornea of the eye by, for example, a contact lens or into the cornea of the eye by, for example, a laser.

Or, alternatively, the second lens construction can comprise two independent elements, firstly, a moving, translating, element comprising a free-form surface and a non-moving, static element comprising the complementary free-form surface. Such static element can be a piggy back element positioned on top of the first lens construction, or, alternatively, the static element can be the cornea of the eye on which a free-form surface is attached, for example by a contact lens or etched into the cornea by, for example, a laser, or, for example, such free-form can be etched on top of a phakic anterior chamber intraocular lens. Or, alternatively, such free form can be added to the, preferably, anterior surface of any, static, intraocular lens, the first lens construction, inside the remains of the capsular bag. Such combination of two spherical optics of one of the optics translated in a direction largely perpendicular to the optical axis will result in a distorted image, by introduction of, for example, coma aberrations due to decentration. However, such aberrations can be minimized by concentration of the main fixed optical provided by the first, stable, lens construction, for example, depending on the requirements of the particular eye, the first lens construction providing 20D of fixed optical power for refractive correction and the second lens construction providing, say, 2.5D variable power for accommodation. With such combination the aberrations at accommodation might not be noticeable to the wearer of the intraocular lens combination.

The second lens construction can comprises mechanical components to provide translation of lateral compression of the construction into mutual translation of the optical elements, or, alternatively, the second lens construction can comprise at least one elastic optical component which component is to provide variable defocus power which power depends on the degree of change of shape of the elastic optical component with the second lens construction comprising mechanical components adapted to provide translation of lateral compression of the construction into a change of shape of the elastic optical component, with the second lens construction to be implanted at the sulcus plane of the eye with the second lens construction comprising at least one mechanical component providing translation of movement of the ciliary mass of the eye into mutual translation of the optical elements, or, alternatively, the second lens construction comprising at least one mechanical component providing translation of movement of the ciliary mass of the eye into a change of shape of the elastic optical component, and, the second lens construction also comprising at least one additional optical surface adapted to provide corrective optical power to correct at least one optical aberration of the eye which can be fixed optical power to correct at least one fixed optical aberration of the eye, which can be residual refractive error of the eye, for example, myopia of the eye, or, hyperopia of the eye, or, astigmatism of the eye, or at least one additional optical surface provides variable optical power to correct at least one variable optical aberration of the eye other than variable defocus which can be variable aspherical aberration with the shape of the posterior optical surface of the second lens construction providing a fit with the anterior surface of the first lens construction.

So, in summary, this document discloses an intraocular lens combination with a first lens construction and a second lens construction with the lens combination providing fixed optical power and variable optical power with the first lens construction providing at least a part of the fixed optical power of the lens combination and that the second lens construction providing at least a part of the variable optical power of the lens combination, or, alternatively, with the first lens construction providing all fixed optical power of the lens combination and that the second lens construction provides all variable optical power of the lens combination.

The first lens construction can comprise a monofocal intraocular lens with the first lens construction implanted inside the capsular bag, and, the second lens construction can comprise a variable intraocular lens with the second lens construction implanted outside the capsular bag of the eye.

The second lens construction can comprise a combination of at least two optical elements comprising a combination of at least two complementary free-form optical surfaces with each optical element comprising at least one such free-form optical surface with the combination adapted to provide a lens of variable defocus power which power depends on the degree of mutual translation of the optical elements in a direction perpendicular to the optical axis of the eye, or, alternatively, the second lens construction can comprise at least one elastic optical component to provide variable defocus power which the optical power depends on the degree of change of shape of the elastic optical component.

Also, at least one of the lens constructions can comprise at least one additional optical surface to provide correction of at least one residual optical aberration of the eye.

The method for implantation of such an intraocular lens combination can comprise multiple steps including, firstly, replacement of the natural lens by a first lens construction, and secondly, after a period of time, post operative, evaluation of residual fixed and variable aberrations of the eye and, thirdly, implantation of a second lens construction adapted to provide a combination of accommodation and correction of any number of residual optical aberrations.

1. An intraocular lens combination comprising a first lens construction and a second lens construction wherein the lens combination provides fixed optical power and variable optical power with the first lens construction providing at least a part of the fixed optical power of the lens combination and that the second lens construction providing at least a part of the variable optical power of the lens combination.

2. The intraocular lens construction as claimed in claim 1, wherein the first lens construction provides all fixed optical power of the lens combination and that the second lens construction provides all variable optical power of the lens combination.

3. The intraocular lens combination according to claim 1, wherein the first lens construction comprises a monofocal intraocular lens with the first lens construction adapted to be implanted inside the capsular bag.

4. The intraocular lens combination according to claim 1, wherein the second lens construction comprises a variable intraocular lens with the second lens construction adapted to be implanted outside the capsular bag of the eye.

5. The intraocular lens combination according to claim 4, wherein the second lens construction comprises a combination of at least two optical elements comprising a combination of at least two complementary free-form optical surfaces with each optical element comprising at least one such free-form optical surface with the combination adapted to provide a lens of variable defocus power which power depends on the degree of mutual translation of the optical elements in a direction perpendicular to the optical axis of the eye.

6. The intraocular lens combination according to claim 4, wherein the second lens construction comprises at least one elastic optical component adapted to provide variable defocus power which power depends on the degree of change of shape of the elastic optical component.

7. The intraocular lens combination according to claim 1, wherein at least one of the lens constructions also comprises at least one additional optical surface adapted to provide correction of at least one residual optical aberration of the eye.

8. A method for implantation of an intraocular lens combination comprising a first lens construction and a second lens construction with the first lens construction providing at least a part of the fixed optical power of the lens combination and the second lens construction providing at least a part of the variable optical power of the lens combination wherein the method includes multiple steps including, firstly, replacement of the natural lens by a first lens construction, and secondly, after a period of time, post-operative, evaluation of residual fixed and variable aberrations of the eye and, thirdly, implantation of a second lens construction adapted to provide a combination of accommodation and correction of any number of residual optical aberrations.
